(19) United States
(12) Patent Application Publication
(10) Pub. No.: US 2009/0263447 A1
(43) Pub. Date: Oct. 22, 2009

(54) Crosslinked hyaluronic acid and process for the preparation thereof

(75) Inventors: Jerome Asius, Baillargues (FR); Nicolas Riviere, Pujaut (FR); Benedicte Asius, Baillarques (FR)

Correspondence Address: THE NATH LAW GROUP, 112 South West Street, Alexandria, VA, 22314, US

(73) Assignee: Stiefel Laboratories, Inc., Coral Gables, FL (US)

(21) Appl. No.: 12/318,699

(22) Filed: Jan. 6, 2009

Related U.S. Application Data
(63) Continuation of application No. PCT/FR2007/052245, filed on Oct. 25, 2007.
(60) Provisional application No. 61/019,503, filed on Jan. 7, 2008.

(30) Foreign Application Priority Data
Nov. 10, 2006 (FR) 0609866

(51) Int. Cl. A61F 2/00; C07H 5/06; C07H 21/00

(52) U.S. Cl. 424/423; 536/29.1; 536/25.3

(57) ABSTRACT

The present invention relates to a crosslinked hyaluronic acid that can be obtained according to a process comprising: (a) activation of a hyaluronic acid, (b) reaction of the activated hyaluronic acid with an oligopeptide- or polypeptide-based crosslinking agent, in a reaction medium adjusted to a pH of from 8 to 12, so as to obtain a crosslinked hyaluronic acid, (c) adjustment of the pH of the reaction medium to a value ranging from 5 to 7, and (d) precipitation of the crosslinked hyaluronic acid from an organic solvent.

It also relates to the above process, to a hydrogel obtained from the crosslinked hyaluronic acid and to the use of the crosslinked hyaluronic acid for the manufacture of implants that can be used in particular in plastic surgery.

The present invention relates to a novel crosslinked hyaluronic acid and also to the process for the preparation thereof and to the uses thereof, in particular cosmetic uses.

Hyaluronic acid is a polysaccharide consisting of D-glucuronic acid units and N-acetyl-D-glucosamine units, which is in particular known to be used in repair surgery or ocular surgery or else in the aesthetics field as a product for filling wrinkles. In the latter application, in particular, hyaluronic acid is preferred to other filling products due to its biocompatibility and its physicochemical properties. However, it has the drawback that it degrades rapidly, thus requiring repeated injections. In order to remedy this disadvantage, various processes for crosslinking hyaluronic acid, aimed at making it less sensitive to the various degradation factors, such as enzymatic and/or bacterial attacks, temperature and free radicals, and thus at improving its resistance to degradation in vivo and consequently its duration of action, have been proposed. These processes involve, in particular, an etherification, an esterification or an amidation of the hydroxyl and/or acid functions of natural hyaluronic acid.

The prior art processes for crosslinking hyaluronic acid, in particular by amidation, have, however, the drawback of producing hyaluronic acid derivatives that are difficult to formulate and to syringe in an aqueous medium and/or insufficiently resistant to degradation factors, in particular after sterilization of the product.

This is true of the water-insoluble hyaluronic acid prepared according to application US 2001/0393369 by reaction, in an acidic medium, of hyaluronic acid with an activating agent such as 1-ethyl-3-(3-dimethylaminopropyl)carbodiimide (EDC) and a nucleophile which may be a polylysine.

It is in fact thought that, at pHs below or equal to 7, the expected amidation reaction competes with an intramolecular esterification reaction which results in self-crosslinking of the primary alcohol carried by the hyaluronic acid on the activated hyaluronic acid ester. This parasitic reaction is in particular reflected by a considerable increase in the viscosity (solidification) and opacification of the reaction mixture, which is thus in the form of a heterogeneous mixture of water and of insoluble polymer. It then becomes impossible to formulate the hyaluronic acid obtained.

In addition, application EP-1 535 952 discloses a coating consisting of crosslinked hyaluronic acid formed in situ by reaction of a polylysine with hyaluronic acid in the presence of EDC and NHS at a pH of from 2 to 9, and preferably from 4 to 7.5. The article provided with this coating may in particular be a prosthesis that can be used in aesthetic surgery. This document does not disclose crosslinked hyaluronic acid precipitated in an organic solvent with a view to being available in dry form and thus capable of being formulated in the form of a hydrogel in an extemporaneous manner.

Moreover, U.S. Pat. No. 6,630,457 describes a modified hyaluronic acid prepared by reaction of a primary amine on a hyaluronic acid activated with a carbodiimide such as EDC and an N-hydroxysulphosuccinimide derivative such as NHS, at a pH of from 7.0 to 8.5. The compound obtained can be crosslinked under physiological conditions, for example, with glutaraldehyde, so as to obtain a hydrogel which remains sensitive to glycosidases and degrades substantially entirely in less than 50 hours. These degradation kinetics are compatible with the envisaged use as a vector for cells and for growth factors, but is not suitable for use as a filling material in aesthetic surgery, for example.

Finally, application WO 2006/021644 describes a process for preparing crosslinked hyaluronic acid by activation of hyaluronic acid with a coupling agent such as EDC and a catalyst such as NHS, followed by a reaction with a polypeptide such as dilysine, at a pH of from 4 to 10, for example, from 4 to 6. The pH can optionally be increased, at the end of the reaction, to a value of from 6 to 7 in order to increase the yield from the extraction during the precipitation phase. Thus, either the crosslinking is carried out in an acidic medium which is then optionally neutralized, or it is carried out in a basic medium without subsequent pH modification.

The applicant has discovered that the use of an acidic pH during the reaction phase is not always favourable to the amidation reaction and can, as indicated above, result in parasitic reactions, in particular intramolecular esterification reactions, capable of affecting the physicochemical properties of the product obtained.

There remains therefore the need to propose a crosslinked hyaluronic acid that can be obtained in dry form and then readily reformulated in an aqueous medium so as to form a hydrogel having good physicochemical properties, reflected in particular by an elastic modulus G and a loss angle delta of less than 30, which hydrogel is itself capable of being subjected to a thermal treatment, in particular a sterilization treatment, with a view to being used for the manufacture of an implant that is itself sufficiently stable with respect to the various degradation factors such as enzymatic and/or bacterial attacks, temperature and free radicals, so as not to completely resorb in vivo in less than 4 months.

Now, the applicant has discovered, entirely fortuitously, that the pH of precipitation, from an organic solvent, of the hyaluronic acid crosslinked with a polypeptide determines its rheological properties and its sensitivity with respect to degradation factors such as temperature, free radicals and enzymes such as hyaluronidases. Following many experiments, the applicant has subsequently identified the optimal precipitation conditions with a view to obtaining a crosslinked hyaluronic acid relatively insensitive to thermal degradation, i.e. conserving its rheological properties after resolubilization of the precipitated compound and sterilization. It is thus as if the crosslinked hyaluronic acid, once reformulated, conserves a “memory” of its molecular organization at the time of the precipitation. It has, moreover, been demonstrated that this molecular arrangement also has an influence on the ability of the polymer to be resolubilized.

Without wishing to be bound by this theory, it is thought that the abovementioned process makes it possible to densify and solidify the macromolecular network of the hyaluronic acid, not only by means of covalent bonds with the crosslinking agent, but also by means of ionic interactions and/or hydrogen bonds that develop at the time of the precipitation.

A subject of the present invention is therefore a crosslinked hyaluronic acid that can be obtained according to a process comprising:
activation of a hyaluronic acid using a coupling agent and an auxiliary coupling agent, so as to obtain an activated hyaluronic acid,
reaction of the activated hyaluronic acid with a crosslinking agent comprising at least 50% by weight of oligopeptide or polypeptide, in a reaction medium adjusted to a pH of from 8 to 12, so as to obtain a crosslinked hyaluronic acid,
adjustment of the pH of the reaction medium to a value ranging from 5 to 7,
precipitation of the crosslinked hyaluronic acid from an organic solvent so as to obtain fibres of crosslinked hyaluronic acid, and
optionally, drying of the fibres of crosslinked hyaluronic acid obtained.

The crosslinked hyaluronic acid obtained according to the invention is water-soluble. This expression is intended to mean that 1 g of the dehydrated fibres obtained as described above disaggregate in a few minutes and are completely solubilized in one litre of physiological saline solution after a few hours without stirring.

The hyaluronic acid used in the process above is generally used in the natural state, i.e. as it is naturally present in a living organism or excreted by the bacteria when it is produced by bacterial fermentation. It thus generally has a molecular mass ranging from 500 000 to 7 000 000 Daltons and is normally used in the form of a sodium salt.

The hyaluronic acid is activated before crosslinking, using a coupling agent and an auxiliary coupling agent.

Examples of coupling agents are water-soluble carbodiimides such as 1-ethyl-3-(3-dimethylaminopropyl)carbodiimide (EDC), 1-ethyl-3-(3-trimethylaminopropyl)carbodiimide (ETC) and 1-cyclohexyl-3-(2-morpholinoethyl)carbodiimide (CMC) and also salts thereof and mixtures thereof. EDC is preferred for use in the present invention.

Examples of auxiliary coupling agents are N-hydroxysuccinimide (NHS), N-hydroxybenzotriazole (HOBt), 3,4-dihydro-3-hydroxy-4-oxo-1,2,3-benzotriazole (HOOBt), 1-hydroxy-7-azabenzotriazole (HAt) and N-hydroxysulphosuccinimide (sulpho-NHS), and mixtures thereof. Without being limited to the choice of NHS, the latter is preferred for use in the present invention.

The role of the agent and of the auxiliary coupling agent is illustrated in Example 1, hereinafter.

According to the invention, it is preferred for the molar ratio of the coupling agent to the carboxylic acid units of the hyaluronic acid to be between 2% and 200%, more preferably between 5% and 100%.

In addition, the molar ratio of the auxiliary coupling agent to the coupling agent is advantageously between 1:1 and 3:1, preferably between 1.5:1 and 2.5:1, limits inclusive, and more preferably equal to 2.

The reaction for activation of the hyaluronic acid with the coupling agent can be carried out at a pH ranging, for example, from 3 to 6, preferably from 4 to 5.

The concentration of hyaluronic acid in the reaction medium is, for example, between 0.1% and 5% by weight, for example between 0.1% and 1% by weight, limits inclusive.

The crosslinking agent comprises at least 50% by weight, and advantageously consists, of an oligopeptide or polypeptide which may be a random, block, segmented, grafted or star homo- or copolypeptide. The cross-linking agent is generally in the form of a salt, and in particular in hydrochloride or optionally hydrobromide or especially trifluoroacetate form.

Examples of polypeptides that can be used in the present invention are lysine, histidine and/or arginine homo- and copolymers, in particular polylysines having at least two, or even at least five, lysine units, such as dilysine, polyhistidines and polyarginines, without this list being limiting. These amino acids can be in D form and/or in L form. Dilysine and salts thereof, and also derivatives thereof, are preferred for use in the present invention.

According to the invention, it is preferred for the number of amine functions of the polypeptide involved to represent from 1% to 100%, preferably from 10% to 50%, of the number of carboxylic acid functions of the hyaluronic acid involved.

In a first preferred variant of the invention, the coupling agent is used in a stoichiometric amount relative to the amine functions of the crosslinking agent. In this manner, at the end of the first step of the process according to the invention, the amount of carboxylic acid functions of the hyaluronic acid which are activated is equal to the amount of amine functions which will be added in the second step.

In a second variant of the invention, the coupling agent is used in a stoichiometric amount relative to the carboxylic acid functions of the hyaluronic acid. In this case, at the end of the first step of the process according to the invention, all the carboxylic acid functions of the hyaluronic acid are activated and the amount of crosslinking agent used in the second step may, for example, be less than 30%, better still less than 10%, or even approximately 5% (by number of moles of crosslinking agent relative to the number of moles of carboxylic acid functions).

The crosslinking reaction is generally carried out under temperature conditions and for a period of time that are entirely conventional for those skilled in the art, for example, at a temperature of 0-45° C., preferably 5-25° C. for 1 to 10 h, preferably 1 to 6 h. In order to promote the formation of amide bonds, the pH of the reaction is between 8 and 12, and preferably between 8 and 10 (limits inclusive). This pH can be adjusted using any base, preferably a weakly nucleophilic base, for instance an amine such as diisopropylethylamine (DIEA).

This reaction is normally carried out in a solvent such as an aqueous solution of sodium chloride.

The concentration of hyaluronic acid in the reaction medium is, for example, between 0.01% and 5% by weight, for example between 0.1% and 1% by weight, limits inclusive.

After reaction, the pH of the reaction medium is adjusted to a value ranging from 5 to 7, and preferably from 5.5 to 7, using any acid such as hydrochloric acid, before the crosslinked hyaluronic acid obtained is precipitated. The precipitation step is carried out in an organic solvent such as ethanol, isopropanol, ether or acetone, or mixtures thereof, for example, ethanol being preferred in this invention. The solvent is advantageously used in an amount representing from 5 to 20 times, for example, approximately 10 times, the volume of reaction medium.

An optional drying step is then preferably carried out, so as to obtain a dehydrated form of crosslinked hyaluronic acid which is easier to handle and can be stored more successfully. The storage can, in particular, be carried out under negative cold conditions.

The subject of the invention is also the process for producing a crosslinked hyaluronic acid, as described above.

This process may also comprise steps other than those explicitly mentioned, and in particular a step of mixing said dehydrated crosslinked hyaluronic acid with an aqueous solvent, such as a sodium chloride solution, a physiological saline solution or a buffered solution that is injectable (in particular a phosphate buffered saline solution), so as to form a hydrogel. The concentration of hyaluronic acid in said hydrogel can range from 1% to 4%, and preferably from 1.5% to 3% by weight/volume.

A subject of the invention is therefore also such a hydrogel, containing a crosslinked hyaluronic acid as described above, in an aqueous solvent.

The hydrogel thus obtained has, after sterilization, for example at 118-130° C. for 2 to 30 minutes, in accordance with the invention, an elastic modulus G″ of at least 100 and, for example, between 200 and 600 Pa, limits inclusive, and a variation in its elastic modulus of less than 30%, and preferably of less than 20%, after stoving at 93° C. for 1 hour. It also advantageously has a viscous modulus G″ ranging from 50 to 200 Pa; a loss angle δ [=Inv tan (G″/G′)] ranging from 15 to 35° and a viscosity η ranging from 1000 to 3000 Pa·s. The measurement of the elastic modulus, of the viscous modulus and of the loss angle can be carried out in the following way: the hydrogel is treated with a cone-plate geometry of 4 cm, 40 at a temperature of 25° C. It is subjected to a non-destructive viscoelastic test at 1 Hz, with an imposed deformation of 1%. The measurement of the elastic modulus is carried out using an AR 1000 rheometer from the company TA Instruments. The same apparatus can be used for measuring the viscosity using a shear gradient of 5×10⁻² sec⁻¹.

A subject of the invention is thus also a sterilized hydrogel containing hyaluronic acid crosslinked with a crosslinking agent containing at least 50% by weight of oligopeptide or polypeptide, characterized in that it exhibits a variation in its elastic modulus of less than 30% after stoving at 93° C. for 1 hour.

This hydrogel is advantageously used for the manufacture of implants.

These implants can in particular be injected subcutaneously (hypodermally) or intradermally into the fibrous tissue.

They may contain, in addition to the abovementioned hydrogel, a vector fluid comprising at least one polysaccharide, for example, at least one cellulose derivative such as carboxymethylcellulose and/or at least one glycosaminoglycan such as a sodium hyaluronate and/or particles of a biocompatible, bioresorbable material such as polylactic acid (PLA), polyglycolic acid (PGA), poly(lactic-co-glycolic) acids (PLGA), tricalcium phosphate (TCP), or hydroxyapatite (HAP), and mixtures thereof.

Examples of such minerals of implants containing them are in particular described in application WO 2004/069090.

The implants according to the invention are bioresorbable, in the sense that they are capable of degrading in the organism in 6 to 18 months.

They may in particular be used for:
supplementation of a cavity or organ deficient in hyaluronic acid (typically in dermatology, in aesthetic medicine or in orthopaedic treatments);
reconstitution of a volume effused during surgical interventions (typically in ocular surgery), or
topical application to the normal or damaged dermis (typically in cosmetology and dermatology).

The abovementioned implant is particularly suitable for use in filling facial wrinkles and fine lines and/or scars on the human body.

A subject of the present invention is therefore also the use of the crosslinked hyaluronic acid as described above for the manufacture of injectable implants for use in aesthetic and/or repair surgery, or for the manufacture of filling products, in particular products for filling wrinkles, fine lines, scars or depressions of the skin, such as lipodystrophies.

The invention will now be illustrated by the following non-limiting examples.

### EXAMPLES

### Example 1

### Synthesis of Hyaluronic Acid Crosslinked with a Polypeptide According to the Invention

### 1. Reaction Scheme

The reaction scheme followed can be illustrated in the following way (taking dilysine as example):

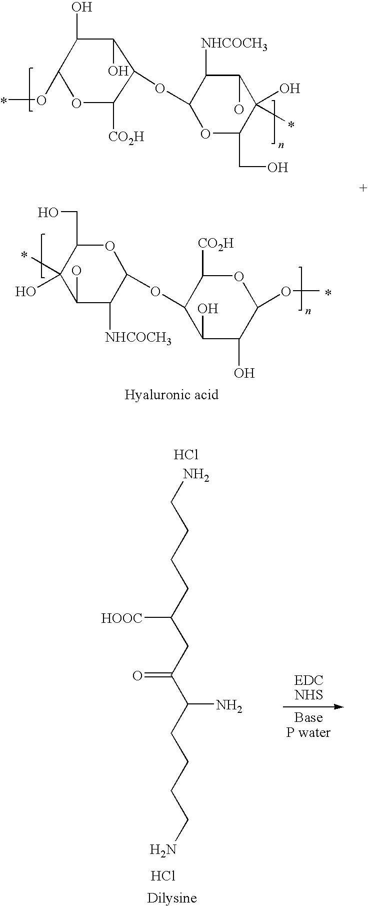

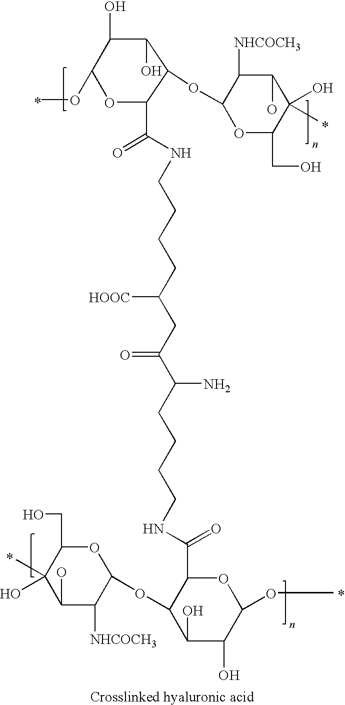

The crosslinking reaction (scheme 1) consists of a double peptide coupling between the carboxylic acid functions of two hyaluronic acid chains and the amine functions of dilysine. The coupling reagents used are 1-ethyl-3-(3-dimethylaminopropyl)carbodiimide (EDC) and N-hydroxysuccinimide (NHS).

The mechanism of the coupling reaction can be illustrated in the following way:

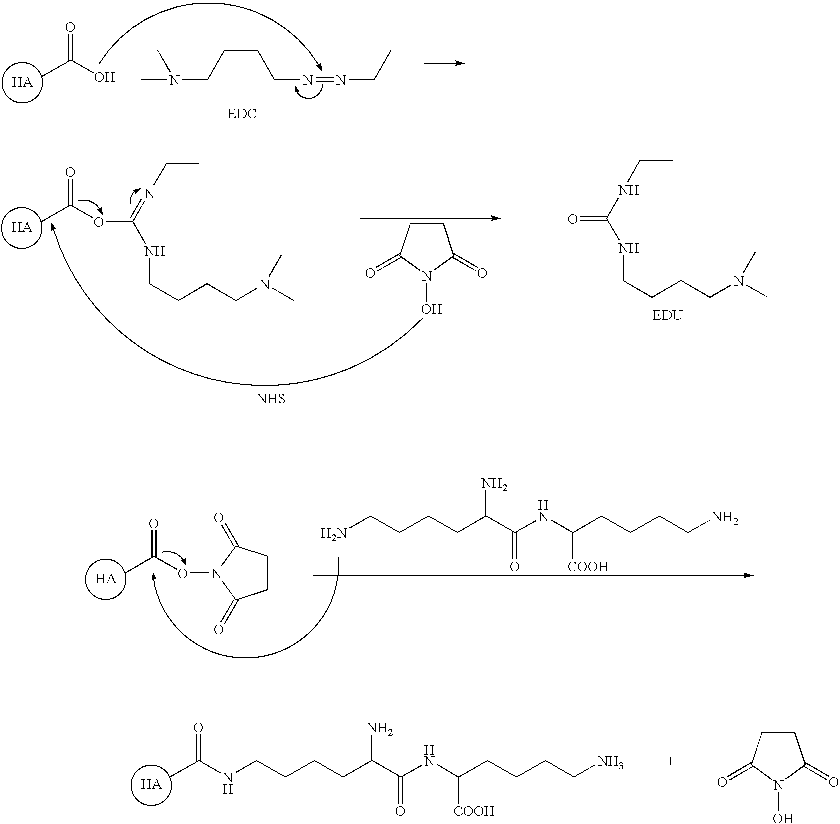

The first step consists of a nucleophilic attack by the carboxylic acid function of the hyaluronic acid on the carbodiimide function of the EDC coupling agent. The resulting O-acylurea is then substituted with NHS so as to form a more stable activated ester (production of 1-ethyl-3-(3-dimethylaminopropyl)urea). In fact, the O-acylurea can become rearranged to inert N-acylurea in a slightly acidic aqueous medium and during a long reaction time. The latter step consists, finally, of the nucleophilic attack by one of the amine functions of the dilysine (preferably terminal, sterically favoured) on the activated ester in order to form an amide bond with release of NHS.

### 2. Protocol

### 1st Step: Swelling Phase

3 g of sodium chloride are successively added to 300 ml of milliQ water in a 500 ml glass reactor. After dissolution of the sodium chloride in a sonicator, 2 g of hyaluronic acid (HTL Sarl, batch No. PH 1016, Mw=2.6×10⁶ Daltons, hereinafter referred to as HA) are introduced into the reactor containing the saline solution, taking care to fray the HA fibres as much as possible by hand. After having stirred the heterogeneous medium with a spatula for 1 minute, the reactor is placed at 4° C. for 15 h without stirring and covered with aluminium foil so as to protect the reaction medium.

### 2nd Step: Crosslinking Phase

The reaction mixture is removed from the refrigerator and then stirred at ambient temperature (18-25° C.) for 10 minutes (visually, the solution should be completely clear and homogeneous, having a certain viscosity, such as fluid honey).

The stirring is of the mechanical type with a half-moon-shaped Teflon stirrer. The rotation rate is 60 rpm.

Next, a solution of 464 mg (4.03 mmol) of N-hydroxysuccinimide (Acros, 98% purity, hereinafter referred to as NHS) in 5 ml of milliQ water is prepared in a haemolysis tube and is then vortexed so as to dissolve all the NHS. This solution is added to the reaction medium dropwise at a rate of 5 ml/min.

The mixture is left to stir for 5 minutes and then a solution of 313 mg (2.02 mmol) of N-(3-dimethylaminopropyl)-N-ethylcarbodiimide hydrochloride (Sigma-Aldrich, ref. 03450-5G, hereinafter referred to as EDC) in 4 ml of milliQ water is added. The dissolution is carried out with a vortex and then the addition is carried out dropwise at a rate of 5 ml/min.

The mixture is left to stir for thirty minutes and then the aqueous solution of dilysine is added to the reaction medium at a rate of 1 ml/min. This solution is prepared by solubilizing, by vortexing, in 1 ml of milliQ water, 233 mg (0.67 mmol) of dilysine hydrochloride (supplier Bachem, ref. G2675), and then 1302 μl (10.08 mmol) of diisopropylethylamine (supplier Acros ref. 115225000, hereinafter referred to as DIEA), the whole being in a haemolysis tube. This mixture has two distinct phases forming a reversible emulsion after vigorous stirring. An attempt is made to mix the emulsion as much as possible while it is added to the reaction medium. The pH of the reaction medium should be between 8.5 and 10.5.

The whole is left to stir for 3 h.

### 3rd Step: Purification Phase

After the stirring has been stopped, the pH of the solution is adjusted before precipitation with 1M HCl so as to decrease it to a pH of 5.7.

A reactor with a volume of one litre equipped with a mechanical stirrer and a rake-shaped stirrer rod, is then prepared. 420 ml of 95° ethanol are poured into this reactor and the mechanical stirring is turned on at very high speed (approximately 1000 rpm).

42 ml of reaction mixture containing the crosslinked hyaluronate are then drawn off using a 50 ml syringe, and are then introduced continuously, as a trickle, into the reactor. The solution should be clear, colourless and quite viscous.

As soon as the addition is complete, the stirring is maintained for a further two minutes. The stirrer rod is then removed from the reactor and the polymer obtained is then unrolled on a frit with a porosity II using a pair of forceps. The polymer is rapidly dried in vacuum flask for a maximum of 15 seconds and is then left to dry in a desiccator under vacuum for a minimum of 12 hours.

The final product should be completely white.

### 4th Step: Reformulation Phase

In order to prepare 10 ml of gel at 2.4%, 240 mg of dried crosslinked polymer were introduced into a standard polypropylene syringe equipped with a capper (at the syringe outlet). 10 ml of buffered** solution are subsequently added to the solid, and the whole is then left to swell at 4° C. for 12 to 15 hours.

After having removed the syringe from the refrigerator, the product is rapidly stirred using a mechanical stirrer, at a speed of 1000 rpm. The stirrer rod used is a stainless steel spoon-shaped laboratory spatula. The duration of the stirring is approximately 5 minutes for this product, but is variable according to the viscosity. The final gel should be colourless and completely homogenous.

### Example 2

### Degradation or Persistence Test

### Principle:

Those skilled in the art are used to carrying out accelerated degradation tests that predict the resistance of a polymer to the various degradation factors in vivo (see in particular FR 2861 734).

In this example, one of these tests, which consists in measuring the rheological characteristics of crosslinked products having been sterilized beforehand and then having been subjected to a heating phase at 93° C. for one hour, was carried out. The percentage loss of the elastic modulus (G′) during the heating is then calculated. The lower this percentage, the more resistant the product is to heat and the more it is considered to be capable also of withstanding the other degradation factors. This test is therefore predictive in terms of the rate of degradation in vivo of the crosslinked hyaluronic acid and therefore of the duration of filling of wrinkles that can be obtained.

### Products Tested:

All the products tested are sterile products.

Several commercially available products were tested, along with:
Product 1, which was a hyaluronic acid obtained as described in Example 1, and
Product 2, which was a hyaluronic acid obtained as described in Example 1, except that 45 mol % of EDC; 90 mol % of NHS and 15 mol % of dilysine, relative to the number of moles of COOH units of the hyaluronic acid, and a DIEA/NHS ratio of 2.22, were used.

### Results:

Table 1 below gives the results obtained for the various crosslinked hyaluronic acids tested.

TABLE 1
Crosslinked hyaluronic acid degradation test

| | | | T1 after 1H00 | | |
|---|---|---|---|---|---|
| | T0 | | at 93° C. | | |
| | Elastic | Loss | Elastic | Loss | |
| Sterile | modulus | angle | modulus | angle | % loss |
| samples | (G′) | (Δ) | (G′) | (Δ) | G′ |
| PERLANE ® | 500 | 8.5 | 360 | 8.4 | 28% |
| JUVEDERM ® | 63.5 | 22 | 43.5 | 25 | 31% |
| 30HV ® | | | | | |
| ESTHELIS | 89 | 25 | 43 | 27 | 52% |
| Basic ® | | | | | |
| Product 1 | 262 | 26 | 224 | 30 | 14% |
| Product 2 | 206 | 25 | 199 | 30 | 3% |

It emerges from this table that the modified hyaluronic acids according to the invention show a smaller drop in their elastic modulus than the commercially available crosslinked hyaluronic acids, which demonstrates that they are more resistant to degradation factors.

### Example 3

### Influence of the Precipitation pH

The physicochemical properties of crosslinked hyaluronic acids synthesized substantially as described in Example 1 and precipitated at various pHs from ethanol were compared. The parameters of the processes for synthesizing these compounds are given in Table 2 below:

TABLE 2
Parameters for synthesizing crosslinked hyaluronic acids

| | | | | | Precip- |
|---|---|---|---|---|---|
| | | | | DIEA/NHS | itation |
| Product | % EDC* | % NHS* | % Dilysine* | ratio | pH |
| Product 1 | 40% | 80% | 13.33% | 2.5 | 5.7 |
| Product A | 40% | 80% | 13.33% | 2.5 | 9.0 |
| Product B | 40% | 80% | 13.33% | 2.5 | 4.0 |
| Product 3 | 100% | 200% | 5% | 2.0 | 5.7 |
| Product C | 100% | 200% | 5% | 2.0 | 4.0 |
| Product 4 | 45% | 90% | 15% | 2.22 | 5.20 |
| Product D | 45% | 90% | 15% | 2.22 | 4.0 |

*relative to the number of moles of carboxylic acid functions of the hyaluronic acid.

The physicochemical properties of the above products were evaluated, once reformulated as described in Example 1, before and after one hour spent in an incubator at 90° C. More specifically, the viscosity of the hydrogel was evaluated and its elastic modulus was measured. The results obtained are given in Table 3 below:

Classes of 1 to 5 were used, which represent a summarizing mark taking into account the elasticity and the viscosity of the gel. The more elastic the gel is considered to be, the higher the mark. Conversely, a non-homogeneous and/or fluid gel has a low mark.

TABLE 3
Physicochemical properties of the crosslinked hyaluronic acids

| | Appearance of | | Appearance of | | % |
|---|---|---|---|---|---|
| | the hydrogel | G′ | the hydrogel | G′ | loss |
| Product | (T0) | (T0) | (T60) | (T60) | G′ |
| Product 1 | Clear, | 262 | Clear, | 224 | 14% |
| | slightly | | elastic | | |
| | granular | | (Class 5) | | |
| | (Class 5) | | | | |
| Product A | Not very | — | — | — | — |
| | viscoelastic | | | | |
| | (Class 2) | | | | |
| Product B | Clear, | — | Clear | — | — |
| | viscous | | (Class 3) | | |
| | (Class 4) | | | | |
| Product 3 | Very clear | 296 | Very clear | 215 | 27% |
| | (Class 5) | | (Class 5) | | |
| Product C | Very clear | — | Very clear | — | — |
| | (Class 5) | | (Class 2) | | |
| Product 4 | Clear, | 206 | Clear, | 199 | 3% |
| | slightly | | elastic | | |
| | granular | | (Class 5) | | |
| | (Class 5) | | | | |
| Product D | Very clear, | — | Not very | — | — |
| | viscoelastic | | viscoelastic | | |
| | (Class 5) | | (Class 2) | | |

It emerges from this table that the crosslinked hyaluronic acids precipitated at basic pH, although easy to reformulate in the form of hydrogels, do not give hydrogels satisfactory for application of a product for filling wrinkles. It is thought that this phenomenon is due to insufficient development of ionic bonds during the precipitation.

Furthermore, the crosslinked hyaluronic acids precipitated at too acidic a pH give hydrogels having a good viscoelasticity (with the proviso of being able to reformulate them, which is not always possible), but which clearly degrade when placed in the incubator and will therefore be sensitive to endogenous degradation factors.

It in fact appears that only a precipitation pH ranging from 5 to 7 makes it possible to readily formulate a homogeneous hydrogel having a very satisfactory visco-elasticity and which is not substantially reduced after a degradation test. This confirms that, in this pH range, the macromolecular network formed by the electrostatic and covalent bonds is optimal for application as a filling material.

1. Crosslinked hyaluronic acid that can be obtained according to a process comprising:
activation of a hyaluronic acid using a coupling agent and an auxiliary coupling agent, so as to obtain an activated hyaluronic acid,
reaction of the activated hyaluronic acid with a crosslinking agent comprising at least 50% by weight of oligopeptide or polypeptide, in a reaction medium adjusted to a pH of from 8 to 12, so as to obtain a crosslinked hyaluronic acid,
adjustment of the pH of the reaction medium to a value ranging from 5 to 7, precipitation of the crosslinked hyaluronic acid from an organic solvent so as to obtain fibres of crosslinked hyaluronic acid, and
optionally, drying of the fibres of crosslinked hyaluronic acid obtained.

2. Hyaluronic acid according to claim 1, characterized in that the coupling agent is chosen from: 1-ethyl-3-(3-dimethylaminopropyl)carbodiimide (EDC), 1-ethyl-3-(3-trimethylaminopropyl)carbodiimide (ETC) and 1-cyclohexyl-3-(2-morpholinoethyl)carbodiimide (CMC), and also salts thereof and mixtures thereof.

3. Hyaluronic acid according to claim 1, characterized in that the auxiliary coupling agent is chosen from: N-hydroxysuccinimide (NHS), N hydroxybenzotriazole (HOBt), 3,4-dihydro-3-hydroxy-4-oxo-1,2,3-benzotriazole (HOOBt), 1-hydroxy-7-azabenzotriazole (HAt) and N-hydroxysulphosuccinimide (sulpho-NHS), and mixtures thereof.

4. Hyaluronic acid according claim 1, characterized in that the molar ratio of the coupling agent to the carboxylic acid units of the hyaluronic acid is between 5% and 100%, limits inclusive.

5. Hyaluronic acid according to claim 1, characterized in that the molar ratio of the auxiliary coupling agent to the coupling agent is between 1:1 and 3:1, limits inclusive.

6. Hyaluronic acid according to claim 1, characterized in that the reaction for activation of the hyaluronic acid with the coupling agent is carried out at a pH ranging from 3 to 6.

7. Hyaluronic acid according to claim 1, characterized in that the polypeptide is a lysine homo- or copolymer.

8. Hyaluronic acid according to claim 7, characterized in that the lysine homopolymer is dilysine.

9. Hyaluronic acid according to claim 1, characterized in that the coupling agent is used in a stoichiometric amount relative to the amine functions of the crosslinking agent.

10. Hyaluronic acid according to claim 1, characterized in that the coupling agent is used in a stoichiometric amount relative to the carboxylic acid functions of the hyaluronic acid.

11. Hyaluronic acid according to claim 10, characterized in that the amount of crosslinking agent used in the second step is less than 30%, by number of moles of crosslinking agent relative to the number of moles of carboxylic acid functions.

12. Hyaluronic acid according to any claim 1, characterized in that the crosslinking reaction is carried out at a pH of from 8 to 10.

13. Hyaluronic acid according to claim 1, characterized in that the precipitation pH ranges from 5 to 7.

14. Hyaluronic acid according to claim 1, characterized in that the organic solvent is ethanol or isopropanol.

15. Process for producing a crosslinked hyaluronic acid, characterized in that it is as described in claim 1.

16. Hydrogel, characterized in that it contains a crosslinked hyaluronic acid as described in claim 1, in an aqueous solvent.

17. Sterilized hydrogel containing hyaluronic acid crosslinked with a crosslinking agent containing at least 50% by weight of oligopeptide or polypeptide, characterized in that it exhibits a variation in its elastic modulus of less than 30% after stoving at 93° C. for 1 hour.

18. Use of the crosslinked hyaluronic acid according to claim 1, for the manufacture of injectable implants for use in aesthetic and/or repair surgery, or for the manufacture of filling products, in particular products for filling wrinkles, fine lines, scars or depressions of the skin.
